(19) United States
(12) Patent Application Publication
(10) Pub. No.: US 2003/0235827 A1
(43) Pub. Date: Dec. 25, 2003

(54) Methods and compositions for monitoring primer extension and polymorphism detection reactions

(75) Inventor: Brian McKeown, Headington (GB)

Correspondence Address: KALOW & SPRINGUT LLP, 488 MADISON AVENUE, 19TH FLOOR, NEW YORK, NY, 10022, US

(73) Assignee: Orchid BioSciences, Inc., Princeton, NJ (US)

(21) Appl. No.: 10/179,826

(22) Filed: Jun. 25, 2002

(51) Int. Cl. C12Q 1/68; C12P 19/34

(52) U.S. Cl. 435/6; 435/91.2

(57) ABSTRACT

The methods of invention include the use of control primers to monitor the efficacy of amplification and/or primer extension reactions, and possible subsequent use of these control products as sizing markers. The methods of the invention are applicable to single reactions as well as to high-throughput and multiplex systems, including array-based technologies. One embodiment of the invention comprises monitoring the efficacy of a reaction for the detection of polymorphisms in the scrapie gene.

### BACKGROUND OF THE INVENTION

Extensive progress in the field of biotechnology over the last two decades has given rise to new and promising routes to the identification and investigation of genomic characteristics in all species. Specifically, advances in nucleic acid synthesis and sequencing have led to the development of the science of genomics. High-throughput sequencing technologies have enabled significant milestones such as the mapping of various genomes, including the human genome. With the ability to rapidly sequence large amounts of DNA, large-scale analysis of genomic characteristics has become possible. Technologies are now evolving to identify and characterize features of genomes pertinent to individual or population-based variations in genotypes that may be used for applications such as identifying an individual's susceptibility to a given disease, identifying characteristics of interest in a gene or a genome, and identifying genetic characteristics that cause or promote disease states. Among the most promising of avenues for characterizing genomic variance in individuals and populations is the analysis and characterization of genetic polymorphisms.

Polymorphisms relate to variances in genomes among different species, for example, or among members of a species, among populations or sub-populations within a species, or among individuals in a species. Such variances are expressed as differences in nucleotide sequences at particular loci in the genomes in question. These differences include, for example, deletions, additions or insertions, rearrangements, or substitutions of nucleotides or groups of nucleotides in a genome.

One important type of polymorphism is a single nucleotide polymorphism (SNP). Single nucleotide polymorphisms occur with a frequency of about 1 in 300 to about 1 in 1,000 base pairs, where a single nucleotide base in the DNA sequence varies among individuals. SNPs may occur both inside and outside the coding regions of genes. It is believed that many diseases, including many cancers, hypertension, heart disease, and diabetes, for example, are the result of mutations borne as SNPs or collections of SNPs in subsets of the human population. Currently, one focus of genomics is the identification and characterization of SNPs and groups of SNPs and how they relate to phenotypic characteristics of medical and/or pharmacogenetic relevance, for example.

A variety of approaches to determining, or scoring, the large variety of polymorphisms in genomes have developed. Although these methods are applicable to many types of genomic polymorphisms, they are particularly amenable to determining, or scoring, SNPs.

One preferred method of polymorphism detection employs enzyme-assisted primer extension. SNP-IT™ (disclosed by Goelet, P. et al. WO92/15712, and U.S. Pat. Nos. 5,888,819 and 6,004,744, each herein incorporated by reference in its entirety) is a preferred method for determining the identity of a nucleotide at a predetermined polymorphic site in a target nucleic acid sequence. Thus, this method is uniquely suited for SNP scoring, although it also has general applicability for determination of a wide variety of polymorphisms. SNP-IT™ is a method of polymorphic site interrogation in which the nucleotide sequence information surrounding a polymorphic site in a target nucleic acid sequence is used to design a primer that is complementary to a region immediately adjacent to the target polynucleotide, but not including the variable nucleotide(s) in the polymorphic site of the target polynucleotide. The primer is extended by a single labeled terminator nucleotide, such as a dideoxynucleotide, using a polymerase, often in the presence of one or more chain terminating nucleoside triphosphate precursors (or suitable analogs). A detectable signal or moiety, covalently attached to the SNP-IT™ primer, is thereby produced.

In some embodiments of SNP-IT™, the oligonucleotide primer is bound to a solid support prior to the extension reaction. In other embodiments, the extension reaction is performed in solution and the extended product is subsequently bound to a solid support. In an alternate embodiment of SNP-IT™, the primer is detectably labeled and the extended terminator nucleotide is modified so as to enable the extended primer product to be bound to a solid support.

Ligase/polymerase mediated genetic bit analysis (U.S. Pat. Nos. 5,679,524, and 5,952,174, both herein incorporated by reference) is another example of a suitable polymerase-mediated primer extension method for determining the identity of a nucleotide at a polymorphic site. Ligase/polymerase SNP-IT™ utilizes two primers. Generally, one primer is detectably labeled, while the other is designed to be bound to a solid support. In alternate embodiments of ligase/polymerase SNP-IT™, the extended nucleotide is detectably labeled. The primers in ligase/polymerase SNP-IT™ are designed to hybridize to each side of a polymorphic site on the same strand, such that there is a gap comprising the polymorphic site. Only a successful extension reaction, followed by a successful ligation reaction, results in production of a detectable signal. This method offers the advantages of producing a signal with considerably lower background than is possible by methods employing only hybridization or primer extension alone.

An alternate method for determining the identity of a nucleotide at a predetermined polymorphic site in a target polynucleotide is described in Söderlund et al., U.S. Pat. No. 6,013,431 (the entire disclosure of which is herein incorporated by reference). In this alternate method, nucleotide sequence information surrounding a polymorphic site in a target nucleic acid sequence is used to design a primer that is complementary to a region flanking, but not including, the variable nucleotide(s) at the polymorphic site of the target. In some embodiments of this method, following isolation, the target polynucleotide may be amplified by any suitable means prior to hybridization to the interrogating primer. The primer is extended, using a polymerase, often in the presence of a mixture of at least one labeled deoxynucleotide and one or more chain terminating nucleoside triphosphate precursors (or suitable analogs). A detectable signal is produced upon incorporation of the labeled deoxynucleotide into the primer.

Due to the large size of many studies that use SNP information, SNP detection must be rapid, amenable to high-throughput and reliable. Reliably interpreting the results of an assay for polymorphism detection or identification using SNP-based applications is an important consideration, particularly when employing multiplex and high-throughput protocols. Size analysis of primer extension products is one method of interpreting results.

Size analysis of labeled primer extension products, particularly in multiplexed protocols, can be problematic. Size analysis generally relies upon detecting fluorescently labeled primer extension products, labeled with a distinct fluorescent label for each of the four nucleotides A, T, G, and C. A fifth fluorescent dye has also been used as an internal lane size standard for assigning a size to an unknown detection product. However, employment of five dyes exploiting the same limited range in the visible spectrum increases the likelihood of spectral overlap. Further, where a dye is present at high concentration, this may result in saturation of the detector and the appearance of inappropriate labeled fragments underlying the intense band. A sizing system employing a fifth dye internal lane size standard, added to detection primer extension products following completion of primer extension, affords no indicator of the success of the initial polymerase chain reaction which generated the amplicon employed in the primer extension reaction. Systems employing a fifth dye marker also cannot be employed to assess the success of the detection primer extension assay in terms of abundance of the detection product present upon analysis.

Further, it may also be the case that presently available sizing standards contain relatively few distinct labeled molecules, resulting in a sparsely populated standard curve. This can result in unacceptable standard deviation for the calculation of the size of a given unknown species during different analyses. Additionally, the need to add an exogenous standard to the products of amplification represents an additional step to the process, exposing the analysis to increased risk of becoming contaminated, or being the source of contamination.

Thus, there is a need in the art of polymorphism detection and identification in a system that provides for the confirmation of amplification, and that provides for accurate detection and identification of polymorphisms, and that can provide for abundance analysis of reaction products, either separately or simultaneously. There is also a need for a more precise means of sizing that would employ standards of known size which lie in very close proximity to the unknowns.

### SUMMARY OF THE INVENTION

In one embodiment, the invention comprises a method of identifying one or more nucleotide bases of a target nucleic acid sequence, comprising: providing the target nucleic acid sequence having a variant nucleotide base and an invariant nucleotide base, providing a control primer capable of hybridizing immediately adjacent to the invariant nucleotide base of the target nucleic acid sequence, providing a detection primer capable of hybridizing immediately adjacent to a variant nucleotide base of the target nucleic acid sequence; allowing the control primer and the detection primer to hybridize to the target nucleic acid sequence; extending the control primer and the detection primer by one or more nucleotide bases in the presence of a polymerizing agent under suitable conditions to allow primer extension to occur; separating the control primer from the detection primer; and identifying one or more nucleotide bases of the target nucleic acid sequence by detecting any extended control and detection primers and separating the extended detection primer from the extended control primer to ensure primer extension has occurred, thereby identifying one or more nucleotide bases of the target nucleic acid sequence.

In another embodiment, the invention comprises a method of monitoring a primer extension reaction or a reaction that generates a target nucleic acid, comprising: providing the target nucleic acid sequence having a variant nucleotide base and an invariant nucleotide base, providing a control primer capable of hybridizing immediately adjacent to the invariant nucleotide base of the target nucleic acid sequence, providing a detection primer capable of hybridizing immediately adjacent to the variant nucleotide base of the target nucleic acid sequence; allowing the control primer and the detection primer to hybridize to the target nucleic acid sequence; extending the control primer and the detection primer by one or more nucleotide bases in the presence of a polymerizing agent under suitable conditions to allow primer extension to occur; separating the control primer and the detection primer from one another; and identifying one or more nucleotide bases of the target nucleic acid sequence by detecting any extended control primer and detection primer and separating the extended detection primer from the extended control primer to ensure primer extension has occurred, and determining the identity of the nucleotide added to the detection primer and the control primer, thereby identifying monitoring the primer extension reaction.

In yet another embodiment, the invention comprises a method of identifying a product of a primer extension reaction, comprising: providing two or more control primers, one or more target nucleic acid sequences and one or more detection primers, wherein the detection primer is capable of hybridizing to an invariant nucleotide sequence immediately adjacent to a polymorphic site on a target nucleic acid sequence or its complement, and wherein both of the one or more control primers hybridize to an invariant sequence on the one or more target nucleic acid sequences that differs from the invariant sequence to which the one or more detection primers hybridize; allowing the one or more control primers and the one or more detection primers to hybridize to one or more target nucleic acid sequences; extending the one or more control primers and the one or more detection primers in the presence of one or more labeled nucleotide bases, in the presence of a polymerizing agent, under conditions sufficient to allow primer extension to occur; separating the control primers from the one or more detection primers; and detecting the one or more detection primers by separating the one or more detection primers from the one or more control primers, thereby identifying the product of the primer extension reaction.

In yet another embodiment, the invention comprises a method of monitoring a primer extension reaction, comprising: amplifying a target nucleic acid sequence from a nucleic acid molecule of interest, in the presence of a polymerizing agent under suitable conditions for amplification to occur, wherein a pair of amplification primers capable of hybridizing to invariant regions of the nucleic acid molecule of interest are employed, and wherein the pair of amplification primers bear at their 5′ ends an invariant tag sequence comprising an invariant base wherein the invariant tag sequence is incapable of hybridizing to the nucleic acid molecule of interest, such that the invariant tag sequence comprising an invariant base is incorporated into a an amplified nucleic acid molecule comprising the target nucleic acid; providing a control primer capable of hybridizing immediately adjacent to the invariant base of the invariant tag sequence in the amplified target nucleic acid, and providing a detection primer capable of hybridizing immediately adjacent to a variant nucleotide base of the amplified target nucleic acid; allowing the control primer and the detection primer to hybridize to the amplified target nucleic acid sequence; extending the control primer and the detection primer by one or more nucleotide bases in the presence of a polymerizing agent under suitable conditions to allow primer extension to occur; separating the control primer from the detection primer; and identifying one or more nucleotide bases of the target nucleic acid sequence by detecting any extended control and detection primers and separating the extended detection primer from the extended control primer to ensure primer extension has occurred, thereby identifying one or more nucleotide bases of the target nucleic acid sequence.

For a better understanding of the present invention together with other and further advantages and embodiments, reference is made to the following description taken in conjunction with the examples, the scope of which is set forth in the appended claims.

### BRIEF DESCRIPTION OF THE FIGURES

Preferred embodiments of the invention have been chosen for purposes of illustration and description, but are not intended in any way to restrict the scope of the invention. The preferred embodiments of certain aspects of the invention are shown in the accompanying figures, wherein:

FIG. 1 illustrates an embodiment of the invention, wherein four control primers are used that hybridize to the same invariant sequence on the target nucleic acid.

FIG. 2 illustrates an embodiment of the invention, wherein target nucleic acid is amplified such that sequences are introduced into the amplicon that can hybridize to control primers.

FIG. 3 illustrates an embodiment of the invention, wherein four distinct regions of interest are co-amplified with amplification primers designed to incorporate exogenous sequences into the amplicons, which exogenous sequences serve as targets for control primers.

FIG. 4 illustrates an embodiment of the invention, wherein the target of the control primer is not part of the amplicon containing the variable base of interest, but is a nucleic acid molecule added post-PCR to the assay, prior to primer extension.

FIG. 5 illustrates an embodiment of the invention, wherein control primers are also employed as flip-back primers such that the extent of self-extension due to flip-back priming can be compared to the extent of extension from a target nucleic acid amplicon.

FIG. 6 illustrates an embodiment of the invention, wherein certain characteristics and behavior of a flip-back primer are shown.

FIG. 7 illustrates features of the most preferred embodiment of the invention.

### DETAILED DESCRIPTION OF THE PREFERRED EMBODIMENTS

The present invention provides methods and compositions for monitoring primer extension reactions and target nucleic acid amplification reactions. Further, the present invention provides methods and compositions that monitor high throughput multiplex detection of polymorphisms.

The figures have been simplified for clarity. For example, the extension product of a primer, which abuts a variant base, is shown as a single peak, as would be the case if the variant position were homozygous. It might be expected that if the variant position was heterozygous, two very closely associated peaks may be generated, with the two extension products having very slightly different mass:charge ratios, due to the different terminal base incorporated, and possibly the different labels attached to the terminating base.

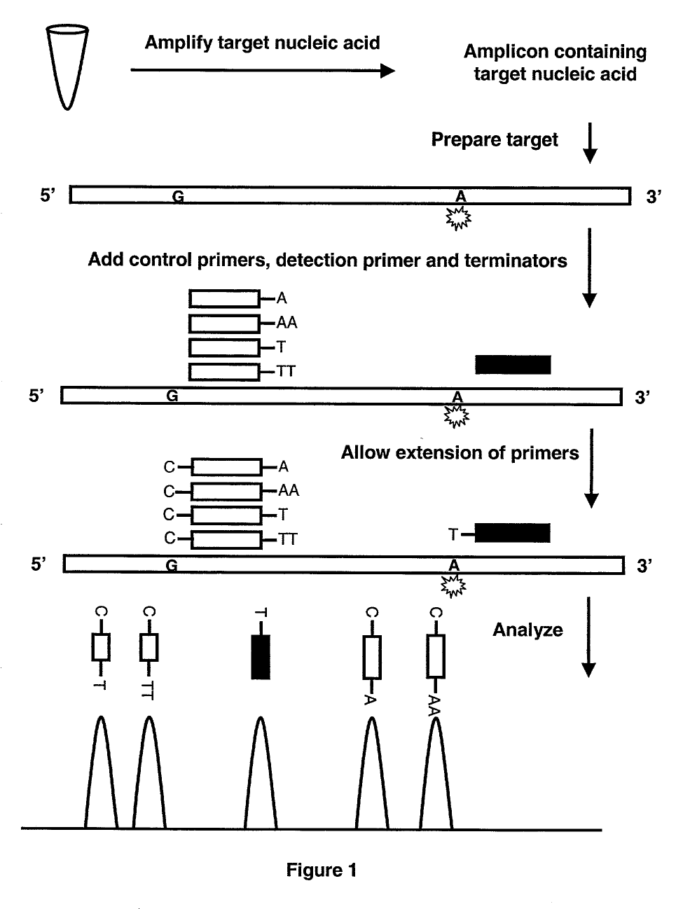

FIG. 1 illustrates certain features of one embodiment of the invention. Target nucleic acid is amplified from a sample, for example, by the polymerase chain reaction. Amplification may not be necessary where ample amounts of target nucleic acid are available. Following amplification, the reaction mixture is prepared for primer extension. Many methods are known in the art to achieve this end, such as, for example, treating the reaction mixture with phosphatases that will inactivate any deoxynucleotides present in the reaction mixture; adding nucleases to remove single stranded primers, then separating or inactivating the phosphatases and nucleases, and other measures known to those skilled in the art. Detection and control primers are then added, along with fluorescently-labeled terminators, and primer extension is allowed to occur. In FIG. 1 four control primers are employed, but more or fewer may be employed. In FIG. 1, all four control primers hybridize to the same invariant region of the target nucleic acid, and are extended by the same invariant residue, “C.” In FIG. 1, the primers differ only by the size (and possibly base composition) of tag sequences at their 5′ ends, where the tag sequences are designed to allow size separation from one another and from the detection primer. The modification at the 5′ end may include additional bases which anneal to the target sequence, although one skilled in the art will appreciate that this will alter the hybridization characteristics of this primer over one with fewer hybridizing bases. Such modifications can be utilized to affect the avidity with which one primer binds, and is therefore extended, in relation to another. In another embodiment, these 5′ extensions could also enable the hybridization of the extended control primers (or detection primers) to specific geographic locations on an array of immobilised DNA with complementary sequence to the specific tags. In FIG. 1, the differences are in the identity and number of nucleotides comprising the tag sequence. Many other kinds of tag sequences can be employed for allowing such separation. Here, the tags shown are selected to separate the primers based on mass:charge ratio. FIG. 1 shows a single detection primer, although the reaction can be carried out in multiplex. FIG. 1 shows the single detection primer hybridizing immediately adjacent to a SNP site, but the variation can be any kind of variation known in the art, such as a deletion, an addition, an insertion, etc. Once the primer extension reaction has occurred, the products of the reaction are analyzed by, for example, a capillary gel electrophoresis apparatus with a fluorescence detector. The apparatus separates the primers based on mass:charge ratio, and the identity of the detection primer can be ascertained by inspecting the distribution of control primers. In FIG. 1, the control primers can be distinguished in fluorescence characteristics from the detection primer, and from each other by mass:charge ratio differences as the result of tag sequence differences. Abundance analysis, sizing algorithms, and the use of flip-back primers are not shown in this example.

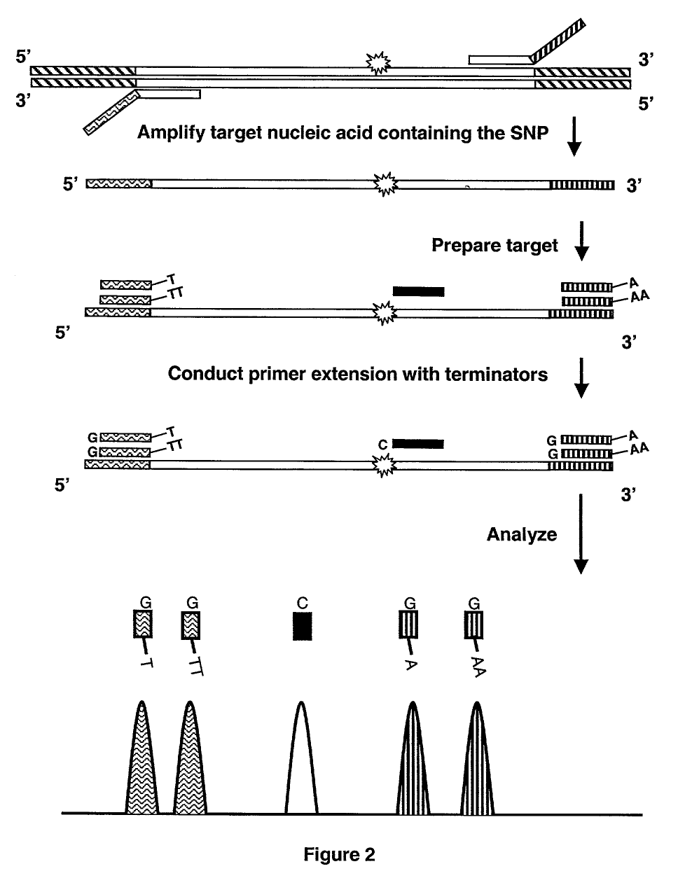

FIG. 2 illustrates certain features of another embodiment of the invention. In FIG. 2, target nucleic acid comprising a variable residue is amplified employing special amplification primers. These amplification primers contain sequences that do not hybridize to sample or target nucleic acids under the selected conditions, but instead contain exogenous sequences that will be incorporated into the amplicon containing the target nucleic acid upon successful PCR amplification of the target. Following amplification, the reaction mixture is prepared for primer extension. Many methods are known in the art to achieve this end, as described above. Detection and control primers are then added, along with fluorescently-labeled terminators, and pnmer extension is allowed to occur. In FIG. 2, four control primers are employed, but more or fewer may be employed. In FIG. 2, the four control primers are targeted such that two hybridize to the same exogenous sequence introduced at one terminus of the amplicon, and two hybridize to the same exogenous sequence introduced at the other terminus. These pairs of control primers may differ in both their core sequence, and the length of any 5′ tag extension. Differences in both core sequence and tag sequence may be used to maximize any differences in mass:charge ratio, whilst maintaining similar hybridization characteristics under given assay conditions. Despite the different targets, as shown in this example, all of the control primers are targeted such that the same invariant base will be incorporated upon successful extension. Here the control primers are extended by the same invariant residue, “G”, although other bases may also be used. In FIG. 2, the pairs of control primers differ both in the core sequence and by the size of tag sequences at their 5′ ends, where the combination of sequence difference and length (and /or base composition) of tag sequences are designed to allow separation from one another and from the detection primer. In FIG. 2, the differences are in the sequence of the pairs of control sequence and the identity and number of nucleotides comprising the tag sequence, although tag sequences alone may be used to alter the characteristics of the control primers where the core sequence of the control primers is identical. Many other kinds of tag sequences can be employed for allowing such separation. Here, the tags shown are selected to separate the primers based on mass:charge ratio. FIG. 2 shows a single detection primer, although the reaction can be carried out in multiplex. FIG. 2 shows the single detection primer hybridizing immediately adjacent to a SNP site, but the variation be any kind of variation known in the art, such as a deletion, an addition, an insertion, etc. Once the primer extension reaction has occurred, the products of the reaction are analyzed by, for example, a capillary gel electrophoresis apparatus with a fluorescence detector. The apparatus separates the primers based on mass:charge ratio, and the identity of the detection primer can be ascertained by inspecting the distribution of control primers. In FIG. 2, the control primers can be distinguished in fluorescence characteristics from the detection primer, and from each other by mass:charge ratio differences as the result of tag sequence differences. Abundance analysis, sizing algorithms, and the use of flip-back primers are not shown in this example.

Referring to FIG. 2 for purposes of illustration, it will be appreciated that the two pairs of control primers hybridizing to two distinct regions of exogenous DNA, one introduced by each of the original amplification primers, can be employed in a variety of ways. The core sequences of the control primers can be designed to be very different so as to, for example, maximize the separation of the extension products of these primers when analyzed by, for example, capillary gel electrophoresis. One example of how differences in control primers can be exploited to advantage is manipulation of the identity of the nucleotides comprising their sequence, and the length of the sequence. For example, if the first pair of control primers is very GC-rich, they might exhibit melting temperatures of 70 degrees Centigrade yet be only 20 and 22 base pairs in length. The second pair of control primers, however, could be very AT-rich, and they would be designed to be, for example, 35 and 37 base pairs in length in order to achieve the same annealing temperature as the first pair of control primers. These differences yield a very large target area for the detection primers to lie between the two pairs of control primers.

It will be appreciated by those of skill in the art, after having read and understood this disclosure, that a large plurality of embodiments employing the control primers taught by this invention can be carried out without undue experimentation. Such embodiments include, for example, singleplex reactions where one variant nucleotide and one invariant control nucleotide are assayed from the same target amplicon, multiplex reactions comprising multiple singleplex reactions amplified and analyzed together where each of the control products contributes to the apparatus for analyzing each of the detection primers, multiplex reactions comprising multiple detection and control primers from the same target amplicon, multiple flip-back primers, and the like. Further, one skilled in the art will appreciate that the introduction of exogenous sequences into the amplicon containing the target nucleic acid affords great versatility in designing control primers. This embodiment of the invention affords the ability to specifically match the qualities of the control primers (such as melting temperature, activity with polymerizing agent, etc.) with the detection primer in a manner that can allow for a high degree of quantitative confidence in the monitoring of the primer extension reaction by, for example, abundance analysis. Similarly, employment of one or more flip-back primers, or employing one or more control primers as flip-back primers, affords the ability to monitor the amplification reaction with a high degree of quantitative confidence. These and other advantages will become apparent to one skilled in the art upon reading and understanding this disclosure.

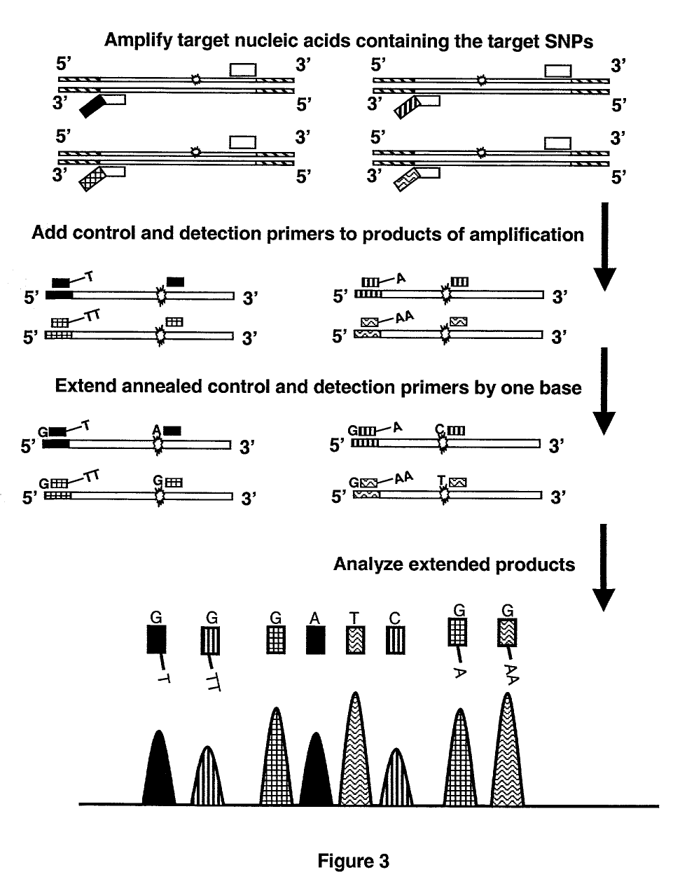

FIG. 3 represents certain features of another embodiment of the invention. In FIG. 3, four distinct regions of interest are co-amplified using amplification primers which are constructed to incorporate at least one exogenous DNA sequence into the amplicon. Each of the amplified regions of interest will in this way generate a control target sequence which can be probed with at least one control primer to generate extension products of known characteristics, both in terms of the base incorporated, which may be the same or different as other control reactions in the same multiplex, and in terms of the reaction kinetics expected of the control reactions under specific reaction conditions.

One skilled in the art will appreciate that through judicious choice of exogenous 5′ sequences attached to the initial amplification primers, large multiplex amplifications can be constructed which will generate control products capable of aiding both the interpretation of individual detection primer reactions, and in the overall interpretation of the multiplex assay, by utilizing the individual control products as components of a sizing ladder for example.

It will be appreciated that through assay of the level of signal returned by a specific control primer, inferences about the relative success of amplification of that particular amplicon within the multiplex shall be possible.

In assays where the same polymorphisms are to be assayed many times, it will be possible to balance the characteristics of a control primer and a detection primer very closely such that correlations of enhanced certainty can be drawn between signal strength of the control reaction and signal strength of the detection reaction. In the absence of such extensive development, one skilled in the art will appreciate that signal strength in primer extension reactions can be a reflection of, at very least, a combination of conditions drawn from: assay conditions, target (amplicon) abundance, extension primer (control and detection) abundance, base incorporated and sequence context around the base incorporated.

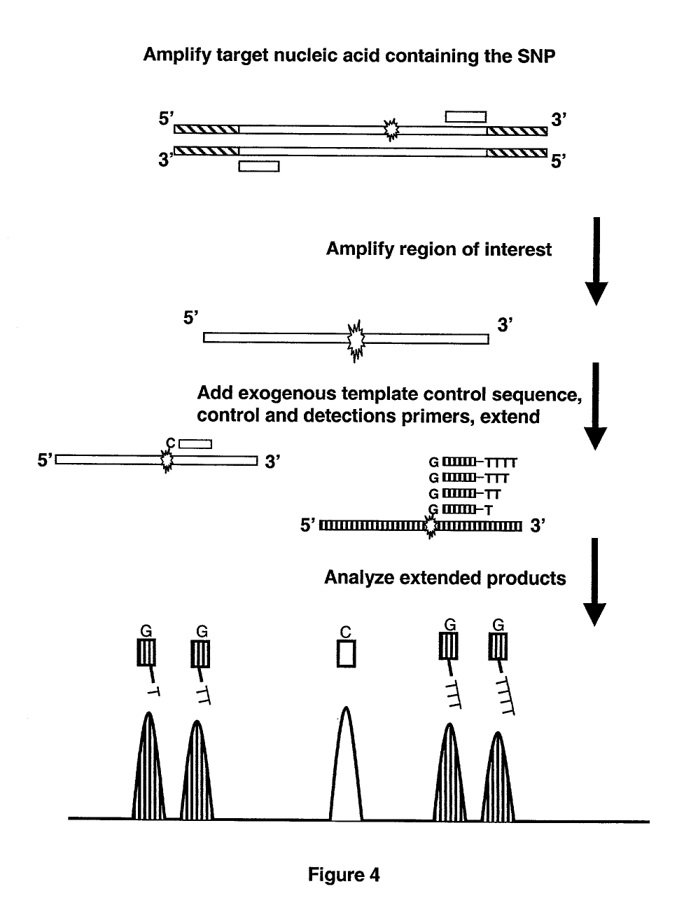

FIG. 4 represents certain features of one embodiment of the invention where the target of the control primer is not part of the amplicon containing the variable base of interested, but is a sequence added post-PCR to the assay, but before primer extension. This system allows for the generation of signal of strength that will intimately reflect the concentration levels of the control target sequence and the control primer under the given assay conditions. Such a system could be used to generate a completely generic control which could be used, as a minimum, as a sizing ladder to allow assay of the extended detection primers present in the assay. One skilled in the art will appreciate that this has advantage where novel detection primers are being used, with unknown electrophoretic migration potential. It is appreciated that short oligonucleotides may migrate under electrophoresis to positions determined not solely by mass:charge, but also the base sequence of the DNA which comprises the oligonucleotide. A sizing ladder which covers a relatively large size range may maximize the potential to be able to size a novel extension product by, for example, a Local Southern algorithm.

FIG. 4 shows all of the control extension products being generated from a single target DNA sequence, but multiple individual exogenous sequences could equally be used, with each one targeted by a single control primer.

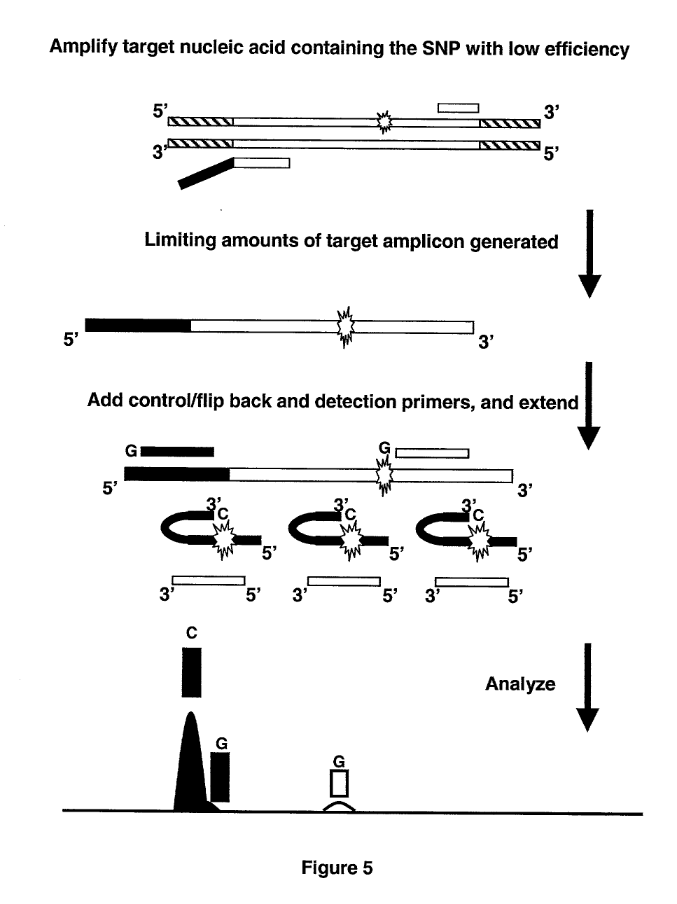

FIG. 5 illustrates certain features of the extension primers, which can either be the control primer or the detection primer. In the example shown, the control primer targets a portion of the amplicon which is generated as a result of successful PCR amplification. In the event that the PCR reaction is unsuccessful, in that it generates a limited amount of target amplicon, the control primers may have the ability to hybridize to themselves with a lower degree of avidity than would be expected from the control primer hybridizing to its fully complementary sequence. One skilled in the art will understand that the propensity for a primer to self anneal at its 3′ terminus will result in primer extension of the primer being supported to some degree, given that there be sufficient base pairing to support the double stranded nature of the DNA for a DNA polymerase to bind to and extend the 3′ terminus by addition of a single nucleotide.

It will be apparent that the base added to the 3′ end of such a flip-back primer will be dependant on the base adjacent to the 3′ terminus of the primer, and that the base added may be the same or different from the base which would have been added had the flip-back primer annealed to an abundant target sequence, which is the preferred situation.

The degree of self-extension compared to extension from an abundant target sequence can be quantifiable, where the self-extension incorporates a base distinct from the normal base added. This can be achieved by, for example, comparison of the amount of each nucleotide incorporated into the control/flip-back primer, as measured by the area under each of the two peaks generated upon electrophoretic separation, or the intensity of the signal generated upon tag capture for each of the two nucleotides.

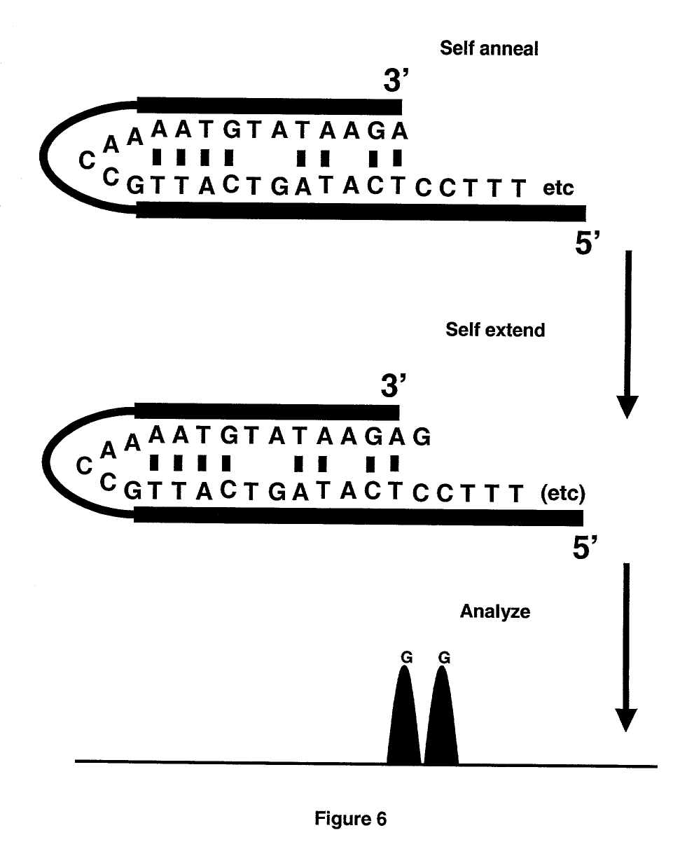

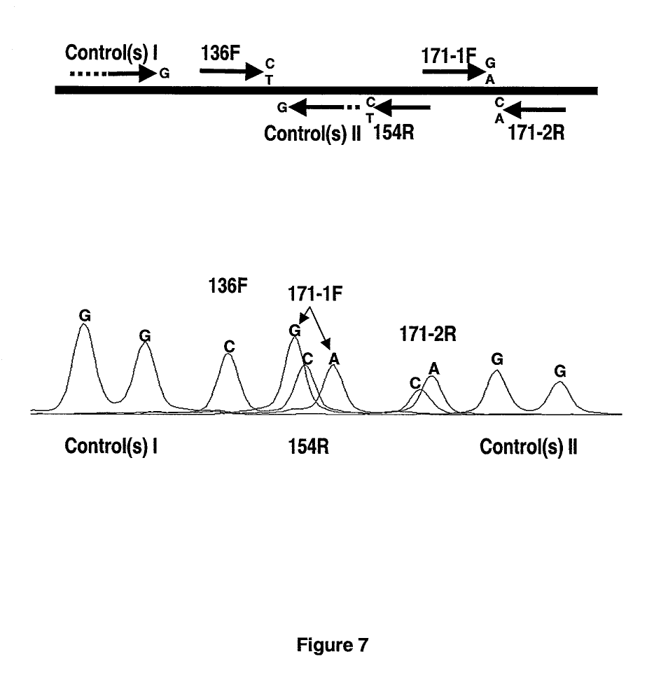

FIG. 6 represents a flip-back primer, as used in the multiplex assay to type four ovine SNPs (see FIG. 7, and Examples below). The primer designed to generate the 36 and 38 bp control products has partial self complementarity and has the ability to support self extension when there is no perfectly homologous template available for it to anneal to (for example, in the case of a failure of PCR to generate an amplicon).

FIG. 7 represents the most preferred embodiment of the invention, which allows the analysis of four SNP sites within the ovine PrP gene sequence. A single 310 bp amplicon is generated by PCR amplification of whole genomic DNA prepared from sheep blood, and control and detection primers hybridize to this amplicon in the approximate positions shown, and in the orientation shown. Primer extension reactions are performed which add a complementary base to the 3′ end of each of the hybridized control and detection primers. When separated under capillary electrophoresis, the fluorescently labeled extended primers separate to form a pattern of peaks which are distinct from one and other on the basis of their size and/or color. The pattern of peaks used in this example profile indicate that the SNP present at 136F was a homozygous C, and also at 154R a homozygous C, whereas the SNP sites at 171-1F and 171-2R are heterozygous GA and heterozygous CA respectively. It will be appreciated that the control peaks are invariant, and will appear in this form regardless of the arrangement of the SNPs present at 136F, 154R, 171-1F and 171-2R. It is further understood that great precision in sizing the SNP site extension products can be gained by ensuring that the control products migrate close to the detection products as shown here. One skilled in the art will appreciate that by addition of non-complementary bases (for example, poly T tails) to the 5′ end of either control or detection primers, the position in which the extension products of these primers migrate under electrophoresis can be subtly altered, as required by the particular assay.

The present invention comprises obtaining a target nucleic acid sequence comprising one or more polymorphisms. The target nucleic acid sequence will preferably be biologically active with regard to the capacity of this nucleic acid to hybridize to an oligonucleotide or a polynucleotide molecule. Target nucleic acid sequences may be either DNA or RNA, single-stranded or double-stranded or a DNA/RNA hybrid duplex. The target nucleic acid sequence may be a polynucleotide or oligonucleotide. Preferred target nucleic acid sequences are between 40 to about 2000 nucleotides in length, in order to facilitate detection. Exceptionally long segments of target nucleic acids, up to several tens of kb, may be required under some circumstances, such as, for example, when analyzing polymorphisms in regions of nucleic acids which have known pseudogenes, and long amplicons are required to enable the selection of amplification primers specific for the gene, rather than the pseudogene. If beneficial, large target nucleic acid sequences may be cut or fragmented into shorter segments by methods known in the art e.g., by mechanical or hydrodynamic shearing methods such as sonication, or by enzymatic methods such as restriction enzymes or nucleases. These shorter segments may then be fractionated such that shorter sequences bearing the polymorphic sites of interest are separated from any redundant sequences that might otherwise participate in undesirable side reactions during analysis of the polymorphisms. Methods of recovering such fractionated DNA are well known in the art, and include gel electrophoresis, HPLC and techniques that capitalize on the recovery of various sequences on the basis of hybridization to a capture sequence.

The target nucleic acid may be isolated, or derived from a biological sample. The term “isolated” as used herein refers to the state of being substantially free of other material such as non nuclear proteins, lipids, carbohydrates, or other materials such as cellular debris or growth media with which the target nucleic acid may be associated. Typically, the term “isolated” is not intended to refer to a complete absence of these materials. Neither is the term “isolated” generally intended to refer to the absence of stabilizing agents such as water, buffers, or salts, unless they are present in amounts that substantially interfere with the methods of the present invention. The term “sample” as used herein generally refers to any material containing nucleic acid, either DNA or RNA or DNA/RNA hybrids. Samples can be from any source including plants and animals including humans. Generally, such material will be in the form of a blood sample, a tissue sample, cells directly from individuals or propagated in culture, plants, yeast, fungi, mycoplasma, viruses, archaebacteria, histology sections, or buccal swabs, either fresh, fixed, frozen, or embedded in paraffin or another fixative. One example of a suitable sample is venous blood taken into a collection device with an anticoagulant such as potassium EDTA. Such a sample is amenable to template preparation by, for example, alkali lysis. Other sample types will be amenable to assay, but may require different or more extensive template preparation such as, for example, by phenol/chloroform extraction, or capture of the DNA onto a silica matrix in the presence of high salt concentration.

Preferably, the target nucleic acids are from genomic DNA drawn from a diverse population so as to do genetic mapping or haplotyping or other studies. Such genomic DNA contains polymorphic site(s) and is used to amplify a region encompassing the polymorphic site(s) of interest through an amplification method such as, for example, the polymerase chain reaction (PCR). Typically the PCR reaction is multiplexed, where two or more or up to 100 or more polymorphic sequences are amplified simultaneously in the same reaction vessel. Preferably, primer extension is carried out in the same reaction as the amplification reaction(s), and preferably sequentially.

The target nucleic acid may be single-stranded and may be derived from either the upper or lower strand nucleic acids of double stranded DNA, RNA or other nucleic acid molecules. The upper strand of target nucleic acids includes the plus strand or sense strand of nucleic acids. The lower strand of target nucleic acids is intended to mean the minus or antisense strand that is complementary to the upper strand of target nucleic acids. Thus, reference may be made to either strand and still comprise the polymorphic site and a primer may be designed to hybridize to either or both strands. Target nucleic acids are not meant to be limited to sequences within coding regions, but may also include any region of a genome or portion of a genome containing at least one polymorphism. The term genome is meant to include complex genomes, such as those found in animals, not excluding humans, and plants, as well as much simpler and smaller sources of nucleic acids, such as nucleic acids of viruses, viroids, and any other biological material comprising nucleic acids. One example of a nucleic acid sequence suitable for analysis is an amplicon from within the coding sequence of the ovine PrP gene, which encodes the prion protein. This protein has known isoforms which can be assayed as the changes in the DNA sequence. A PCR product which comprises these polymorphic sites is a suitable template for assay.

The target nucleic acid sequences or fragments thereof contain the polymorphic site(s), or includes such site(s) and sequences located either distal or proximal to the sites(s). These polymorphic sites or mutations may be in the form of deletions, insertions, re-arrangement, repetitive sequence, base modifications, or single or multiple base changes at a particular site in a nucleic acid sequence. This altered sequence and the more prevalent, or normal, sequence may co-exist in a population. In some instances, these changes confer neither an advantage nor a disadvantage to the species or individuals within the species, and multiple alleles of the sequence may be in stable or quasi-stable equilibrium. In some instances, however, these sequence changes will confer a survival or evolutionary advantage to the species, and accordingly, the altered allele may eventually over time be incorporated into the genome of many or most members of that species. In other instances, the altered sequence confers a disadvantage to the species, as where the mutation causes or predisposes an individual to a genetic disease or defect. As used herein, the terms “mutation” or “polymorphic site” refers to a variation in the nucleic acid sequence between some members of a species, a population within a species or between species. Such mutations or polymorphisms include, but are not limited to, single nucleotide polymorphisms (SNPs), one or more base deletions, or one or more base insertions.

Polymorphisms may be either heterozygous or homozygous within an individual. Homozygous individuals have identical alleles at one or more corresponding loci on homologous chromosomes. Heterozygous individuals have different alleles at one or more corresponding loci on homologous chromosomes. As used herein, alleles include an alternative form of a gene or nucleic acid sequence, either inside or outside the coding region of a gene, including introns, exons, and untranscribed or untranslated regions. Alleles of a specific gene generally occupy the same location on homologous chromosomes. A polymorphism is thus said to be “allelic,” in that, due to the existence of the polymorphism, some members of a species carry a gene with one sequence (e.g., the original or wild-type “allele”), whereas other members may have an altered sequence (e.g., the variant or, mutant “allele”). In the simplest case, only one mutated variant of the sequence may exist, and the polymorphism is said to be biallelic. For example, if the two alleles at a locus are indistinguishable (for example A/A), then the individual is said to be homozygous at the locus under consideration. If the two alleles at a locus are distinguishable (for example A/G), then the individual is said to be heterozygous at the locus under consideration. The vast majority of known single nucleotide polymorphisms are bi-allelic—where there are two alternative bases at the particular locus under consideration. The term “individual” includes an individual of any species, including but not limited to humans.

The present invention utilizes at least one detection primer, at least one control primer and, optionally, a flip-back control primer that can be a control primer as well. The present invention may also utilize two or more amplification primers. In order for an oligonucleotide to serve as a primer, it typically need only be sufficiently complementary in sequence to be capable of forming a double-stranded structure under the conditions employed. Establishing such conditions typically involves selection of solvent and salt concentration, incubation temperatures, incubation times, assay reagents and stabilization factors known to those in the art. The term “primer” or “primer oligonucleotide” refers to an oligonucleotide as defined herein, which is capable of acting as a point of initiation of synthesis when employed under conditions in which synthesis of a primer extension product that is complementary to a nucleic acid strand is induced, as, for example, in a DNA replication reaction such as a PCR reaction. Like non-primer oligonucleotides, primer oligonucleotides may be labeled according to any technique known in the art, such as with radioactive atoms, fluorescent labels, enzymatic labels, proteins, haptens, antibodies, sequence tags, and the like.

Primers can be polynucleotides or oligonucleotides capable of being extended in a primer extension reaction at their 3′ end. As used herein, the term “polynucleotide” includes nucleotide polymers of any number. The term “oligonucleotide” includes a polynucleotide molecule comprising any number of nucleotides, preferably, less than about 200 nucleotides. More preferably, oligonucleotides are between 5 and 100 nucleotides in length. Most preferably, oligonucleotides are 15 to 60 nucleotides in length. The exact length of a particular oligonucleotide or polynucleotide, however, will depend on many factors, which in turn depend on its ultimate function or use. Some factors affecting the length of an oligonucleotide are, for example, the sequence of the oligonucleotide, the assay conditions in terms of such variables as salt concentrations and temperatures used during the assay, and whether or not the oligonucleotide is modified at the 5′ terminus to include additional bases for the purposes of modifying the mass:charge ratio of the oligonucleotide, and/or providing a tag capture sequence which may be used to geographically separate an oligonucleotide to a specific hybridization location on a DNA chip. Short primers may require lower temperatures to form sufficiently stable hybrid complexes with a template. The primers of the present invention should be complementary to the upper or lower strand target nucleic acids. Preferably, the initial amplification primers should not have self complementarity involving their 3′ ends' in order to avoid primer fold back leading to self-priming architectures and assay noise. One exception to the preferred lack of self-complementarity within primers at the 3′ end is that some degree of self-complementarity is preferred when a extension primer is employed as a flip-back primer in an embodiment of the invention. When a primer is to be employed as a flip-back primer, the primer should have sufficient self-complementarity to self-prime in the absence of target nucleic acid, or in the absence of sufficient quantities of target nucleic acid to compete with the self-priming event. Preferred primers of the present invention include oligonucleotides from about 8 to about 40 nucleotides in length, to longer polynucleotides that may be up to several thousand nucleotides long. Preferably, only control primers should be capable of flip-back. Flip-back ability is preferably avoided in amplification and detection primers.

Primers of about 10 nucleotides are the shortest sequence that can be used to selectively hybridize to a complementary target nucleic acid sequence against the background of non-target nucleic acids in the present state of the art. Most preferably, sequences of unbroken complementarity over at least 20 to about 35 nucleotides are used to assure a sufficient level of hybridization specificity, although length may vary considerably given the sequence of the target DNA molecule. The primers of this invention must be capable of specifically hybridizing to the target nucleic acid sequence—such as, for example, one or more upper primers hybridizing to one or more upper strand target nucleic acids or one or more lower strand nucleic acids. As used herein, two nucleic acid sequences are said to be capable of specifically hybridizing to one another if the two molecules are capable of forming an anti-parallel, double-stranded nucleic acid structure or hybrid under conditions sufficient to promote such hybridization, whereas they must be substantially unable to form a double-stranded structure or hybrid with one another when incubated with a non-target nucleic acid sequence under the same conditions. However, in accordance with other embodiments of the invention, when a primer is employed as a flip-back primer, the primer should be capable of self-priming in the absence of sufficient target nucleic acid. For this reason, when a primer is to be employed as a flip-back primer, the primer must possess the ability to self-prime in the absence of sufficient target nucleic acid. Flip-back primers can be designed so that they may incorporate a different nucleotide when extension due to self-priming occurs, as compared to when extension due to priming on the target nucleic acid.

Preferably, when target DNA is absent from the extension reaction, flip-back primers will self extend to some degree, and degree of self extension will be a reflection of the degree of self complementarity and the assay conditions during the extension assay. Flip-back primers will be of greatest utility where there is complete absence of the target amplicon, usually due to complete PCR failure. Preferably, it may be possible to detect very low levels of target amplicon where both self extension of the flip-back primers and desired extension of the flip-back primers are represented by the presence of both extended species, given that the flip-back extension can incorporate a base discrete from that incorporated during the correct control primer extension. Thus, even where a control primer also serves as a flip-back primer, the identity of the extension product will inform as to whether the primer was extended on the amplicon or as the result of flip-back self-priming.

A nucleic acid molecule is said to be the “complement” of another nucleic acid molecule—or itself—if it exhibits complete sequence complementarity. As used herein, molecules are said to exhibit “complete complementarity” when every nucleotide of one of the molecules is able to form a base pair with a nucleotide of the other. “Substantially complementary” refers to the ability to hybridize to one another—or with itself—with sufficient stability to permit annealing under at least under at least conventional low-stringency conditions. Similarly, the molecules are said to be “complementary” if they can hybridize to one another with sufficient stability to permit them to remain annealed to one another under conventional high-stringency conditions. Conventional stringency conditions are described, for example, in Sambrook, J., et al., in Molecular Cloning, a Laboratory Manual, 2nd Edition, Cold Spring Harbor Press, Cold Spring Harbor, N.Y. (1989) (herein incorporated by reference). Departures from complete complementarity are therefore permissible, as long as such departures do not completely preclude the capacity of the molecules to form a double-stranded structure or hybrid. Primers employed as flip-back primers must exhibit sufficient self-complementarity to self-prime in presence of insufficient amounts of target nucleic acid, but preferably will not exhibit complete self-complementarity. Preferably, flip-back primers will have a two to four base pair complementarity at the 3′ end of the flip-back primer. This two to four base pair complementarity need not occur in a single unbroken stretch of self-complementarity. Most preferably, the immediate 3′ terminus will have two bases capable of self hybridization on the primer, followed by two base pairs that are not capable of self hybridization on the primer, followed by two base pairs that are capable of self hybridization on the primer. The actual self complementarity required to generate a flip-back primer will be highly sequence dependant, with G-C pairs being more stable than A-T pairs, and therefore more likely to be able to support self complementarity with fewer matches than a stretch of A-T rich sequence. A single G-C match at the 3′ terminus may be sufficient to support flip-back self-primed extension, even when the adjacent base forms a mismatch.

The primers of the present invention may be tagged at the 5′ end. Tags include any label such as radioactive labels, fluorescent labels, enzymatic labels, proteins, haptens, antibodies, sequence tags, and the like. Preferably, the tag does not interfere with the processes of the present invention. Typically, a tag may be attached to the 5′ end of the primer, with the remainder of the primer sequence being complementary to the target nucleic acid. A preferred tag includes unique tags or marking each type of primer with a distinct sequence that is complementary to a sequence bound to a solid support, where such solid support may include an array, including an addressable array. Thus, when the primer is exposed to the solid support under suitable hybridization conditions, the tag hybridizes with the complementary sequence bound to the solid support. In this way, the identity of the primer can be determined by geometric location on the array, or by other means of identifying the point of association of the tag with the probe. Sequences complementary to the 5′ tag can be bound to a solid support at discrete positions on, for example, an addressable array.

In a preferred embodiment of the invention, one or more control primers bear sequence tags at their 5′ ends that extend the length of the control primers such that their mass:charge ratio differs sufficiently to allow separation based on mass:charge ratio employing methods known in the art, such as, for example, capillary gel electrophoresis. In the most preferred embodiment, four control primers are employed, which can be separated by exploiting differences in their mass:charge ratio from each other and from one or more detection primers. The most preferred embodiment may also include identification of the one or more detection primers by employment of a sizing algorithm such as, for example, a Southern sizing algorithm wherein the control primers are designed to migrate during capillary electrophoresis in pairs: one pair of control primers migrating close together with one another but faster than the one or more detection primers, and a second pair of control primers migrating close together with one another but slower than the one or more detection primers.

Tags can be non-complementary bases, or longer sequences that can be interspersed into the primer provided that the primer sequence has sufficient complementarity with the sequence of the target strand to hybridize therewith for the purposes employed. However, for detection purposes, the detection and control primers in the most preferred embodiment should have exact complementarity to invariant regions of the target nucleic acid(s) to obtain optimal results, where no control primer is employed as a flip-back primer. Thus, primers employed in the present invention must generally be complementary in sequence and be able to form a double-stranded structure or hybrid with a target nucleotide sequence under the particular conditions employed.

An exception to the preference for exact complementarity is whenever a primer is employed as a flip-back primer. In some embodiments of the invention, control primers may also be employed as flip-back primers. When a primer is employed as a flip-back primer, the primer must exhibit sufficient self-complementarity to self-prime in presence of insufficient amounts of target nucleic acid, but preferably will not exhibit complete self-complementarity.

In a preferred embodiment of the invention it is possible to assay the level of extension of the control extension primer, and relate this directly to the level of extension of the detection primer (given that the same base may be incorporated at both sites). One skilled in the art will appreciate that DNA polymerases have, under specific assay conditions, varying propensity to add specific bases to an extending chain dependant on the bases present at the 3′ end of the chain. This is well known from DNA sequencing, where the phenomenon of a poor G incorporation onto an A at the 3′ terminus of a growing chain complicates DNA sequencing interpretation. Such effects might be expected of chain termination primer extension reactions, and so matching control and detection primer sequences at the 3′ terminus can equilibrate the level of extension of each primer under the same or similar assay conditions. Placement of equivalent sequences at the 3′ end of control and detection primers should not render the regions at the 3′ ends identical over a large number of bases. Preferably, at least one base should be identical, but, depending on the assay conditions, it would be useful to limit sequence identity to not more than about three or so bases, as crosstalk between the primers and binding sites may occur with increasing sequence identity, generating erroneous results.

In a preferred embodiment of the invention, analysis of the products of the primer extension reaction can be done so as to determine the relative abundance of labeled control primers, labeled detection primers, and, in some embodiments, labeled flip-back primers. Abundance analysis can be undertaken by comparing the signal strength of the detection primer(s), control primer(s) and flip-back primer(s), and then comparing the relative signal strengths of the primers to determine the relative success of each of the primer extension reactions that occurred. In this way, one skilled in the art can troubleshoot a primer extension reaction, or a combined amplification-primer extension reaction, by examining the relative abundance of the labeled primers. The identity of the incorporated nucleotide or analog thereof into the flip-back primer will be reflected in the extended flip-back primer, and will inform as to the efficiency of the amplification reaction. The ratio of self-primed extension product to target-primed extension product will reflect the abundance of amplified target nucleic acid. The relative abundance of extended extension primer to control primer will inform as to the efficiency of incorporation of the variant nucleotide into the detection primer. In this way, one skilled in the art can learn, in a single reaction run, whether problematic results arose due to sub-optimal amplification, sub-optimal extension of the variant nucleotide, or a host of reaction parameters once the disclosure of this invention is in hand. This embodiment of the invention may be employed to advantage in multiplexed and high-throughput protocols, greatly simplifying troubleshooting of these reactions.

In a preferred embodiment of the invention, amplification primers may be designed to bear specific, known sequences that may or may not reflect sequences found in nature. That is to say, completely artificial sequences may be employed. In this embodiment, amplification primers are designed that are complementary to a target nucleic acid sequence containing one or more polymorphisms of interest. The amplification primers comprise a 5′ tag that is non-complementary to the target nucleic acid to be amplified. The 5′ tag instead is comprised of sequences specifically designed to anneal to sequences comprised in control primers of the present invention. Most preferably, the sequences of the 5′ tag are perfectly complementary to sequences of the control primers employed in a subsequent primer extension reaction. In this way, the amplicon, or amplified sequences of the target nucleic acid, bear the 5′ tag sequences of the amplification primers at one or both termini on the target nucleic acid sequence, or amplicon, comprising the one or more polymorphisms. Most preferably, the 5′ tag sequences that become part of the amplicon are optimized such that they exhibit the same or similar physical characteristics as the invariant region immediately adjacent to the one or more polymorphisms to be detected by the detection primer or primers. By the same or similar physical characteristics is not meant identity of sequence, but rather the same or similar melting temperature or characteristics rendering these sequences about equivalent in their ability to be extended in a primer extension reaction, with respect to the sequence to which the detection primer anneals, as measured by a primer extension reaction. Thus, in one embodiment of the invention, amplification primers may be constructed to introduce, for example, standardized non-natural sequences whose behavior in a primer extension reaction mimic the behavior of the invariant sequences immediately adjacent to the one or more polymorphisms of the target nucleic acid that are to be detected by the detection primer or primers. In the most preferred embodiment, a multiplexed primer extension reaction comprising multiple target nucleic acids are amplified with multiple amplification primers, wherein pairs of amplification primers bear tags that are matched to the control primers employed in the primer extension reaction, thus allowing specific control primers to be employed to monitor the amplification and detection of target nucleic acids comprising specific polymorphisms. In the most preferred embodiment, for each polymorphism to be detected, a unique control primer sequence is employed. Control sequences, in turn, may be detected andlor separated by employing control primers with identifiable 5′ tags. Thus, in an embodiment employing a multiplexed reaction, control primers may be identified and/or separated, for example, by the characteristics of a 5′ tag, the identity of the nucleotide incorporated into the control primer, and/or by the characteristics of the control primers themselves.

Polymerizing agents may be isolated or cloned from a variety of organisms including viruses, bacteria, archaebacteria, fungi, mycoplasma, prokaryotes, and eukaryotes. Preferred polymerizing agents include polymerases. Preferred polymerases for performing single base extensions using the methods and apparatus of the invention are polymerases exhibiting little or no exonuclease activity. More preferred are polymerases that tolerate and are active at temperatures greater than physiological temperatures, for example, at 50° C. to 70° C. or are tolerant of temperatures of at least 90° C. to about 95° C. Preferred polymerases include Taq® polymerase from T. aquaticus (commercially available from ABI, Foster City, Calif.), Sequenase® and ThermoSequenase® (commercially available from U.S. Biochemical, Cleveland, Ohio), and Exo(-) polymerase (commercially available from New England Biolabs, Beverley, Mass.). Any polymerases exhibiting thermal stability may also be employed, such as for example, polymerases from Thermus species, including Thermus aquaticus, Thermus brocianus, Thermus thermophilus, and Thennusflavus; Pyrococcus species, including Pyrococcus furiosus, Pyrococcus sp. GB-D, and Pyrococcus woesei, Thermococcus litoralis, and Thennogata maritime. Biologically active proteolytic fragments, recombinant polymerases, genetically engineered polymerizing enzymes, and modified polymerases are included in the definition of polymerizing agent. It should be understood that the invention can employ various types of polymerases from various species and origins without undue experimentation.

One preferred method of detecting polymorphic sites employs enzyme-assisted primer extension. SNP-IT™ (disclosed by Goelet, P. et al., and U.S. Pat. Nos. 5,888,819 and 6,004,744, each herein incorporated by reference in its entirety) is a preferred method for determining the identity of a nucleotide at a predetermined polymorphic site in a target nucleic acid sequence. Thus, it is uniquely suited for SNP scoring, although it also has general applicability for determination of a wide variety of polymorphisms. SNP-IT™ is a method of polymorphic site interrogation in which the nucleotide sequence information surrounding a polymorphic site in a target nucleic acid sequence is used to design an oligonucleotide primer that is complementary to a region immediately adjacent to, but not including, the variable nucleotide(s) in the polymorphic site of the target polynucleotide. The target polynucleotide is isolated from a biological sample and hybridized to the interrogating primer. Following isolation, the target polynucleotide may be amplified by any suitable means prior to hybridization to the interrogating primer. The primer is extended by a single labeled terminator nucleotide, such as a dideoxynucleotide, using a polymerase, often in the presence of one or more chain terminating nucleoside triphosphate precursors (or suitable analogs). A detectable signal is thereby produced. As used herein, immediately adjacent to the polymorphic site includes from about 1 to about 100 nucleotides, more preferably from about 1 to about 25 nucleotides in the 3′ or 5′ direction of the polymorphic site. Most preferably, the primer is hybridized one nucleotide immediately adjacent to the polymorphic site in the 5′ direction with respect to the polymorphic site.

In some embodiments of SNP-IT™, the primer is bound to a solid support prior to the extension reaction. In other embodiments, the extension reaction is performed in solution (such as in a test tube or a micro well) and the extended product is subsequently bound to a solid support. In an alternate embodiment of SNP-IT™, the primer is detectably labeled and the extended terminator nucleotide is modified so as to enable the extended primer product to be bound to a solid support. An example of this includes where the primer is fluorescently labeled and the terminator nucleotide is a biotin-labeled terminator nucleotide and the solid support is coated or derivatized with avidin or streptavidin. In such embodiments, an extended primer would thus be capable of binding to a solid support and non-extended primers would be unable to bind to the support, thereby producing a detectable signal dependent upon a successful extension reaction.

Ligase/polymerase mediated genetic bit analysis (U.S. Pat. Nos. 5,679,524, and 5,952,174, both herein incorporated by reference) is another example of a suitable polymerase mediated primer extension method for determining the identity of a nucleotide at a polymorphic site. Ligase/polymerase SNP-IT™ utilizes two primers. Generally, one primer is detectably labeled, while the other is designed to be affixed to a solid support. In alternate embodiments of ligase/polymerase SNP-IT™, the extended nucleotide is detectably labeled. The primers in ligase/polymerase SNP-IT™ are designed to hybridize to each side of a polymorphic site, such that there is a gap comprising the polymorphic site. Only a successful extension reaction, followed by a successful ligation reaction, enables production of the detectable signal. The method offers the advantages of producing a signal with considerably lower background than is possible by methods employing either hybridization or primer extension alone.

An alternate method for determining the identity of a nucleotide at a polymorphic site in a target polynucleotide is described in Söderlund et al., U.S. Pat. No. 6,013,431 (the entire disclosure of which is herein incorporated by reference). In this method, the nucleotide sequence surrounding a polymorphic site in a target nucleic acid sequence is used to design an oligonucleotide primer that is complementary to a region flanking the 5′ end, with respect to the polymorphic site, of the target polynucleotide, but not including the variable nucleotide(s) in the polymorphic site of the target polynucleotide. The target polynucleotide is isolated from the biological sample and hybridized with an interrogating primer. In some embodiments of this method, following isolation, the target polynucleotide may be amplified by any suitable means prior to hybridization with the interrogating primer. The primer is extended, using a polymerase, often in the presence of a mixture of at least one labeled deoxynucleotide and one or more chain terminating nucleoside triphosphate precursors (or suitable analogs). A detectable signal is produced on the primer upon incorporation of the labeled deoxynucleotide into the primer.

The primer extension reaction of the present invention employs a mixture of one or more labeled nucleotides and a polymerizing agent. The term “nucleotide” or nucleic acid as used herein is intended to refer to ribonucleotides, deoxyribonucleotides, acyclic derivatives of nucleotides, and functional equivalents or derivatives thereof, of any phosphorylation state capable of being added to a primer by a polymerizing agent. Functional equivalents of nucleotides are those that act as substrates for a polymerase as, for example, in an amplification method or a primer extension method. Functional equivalents of nucleotides are also those that may be formed into a polynucleotide that retains the ability to hybridize in a sequence-specific manner to a target polynucleotide. Examples of nucleotides include chain-terminating nucleotides, most preferably dideoxynucleoside triphosphates (ddNTPs), such as ddATP, ddCTP, ddGTP, and ddTTP; however other terminators known to those skilled in the art, such as, for example, acyclo nucleotide analogs, other acyclo analogs, and arabinoside triphosphates, are also within the scope of the present invention. Preferred ddNTPs differ from conventional 2′deoxynucleoside triphosphates (dNTPs) in that they lack a hydroxyl group at the 3′position of the sugar component.

The nucleotides employed may bear a detectable characteristic. As used herein a detectable characteristic includes any identifiable characteristic that enables distinction between nucleotides. It is important that the detectable characteristic does not interfere with any of the methods of the present invention. Detectable characteristic refers to an atom or molecule or portion of a molecule that is capable of being detected employing an appropriate method of detection. Detectable characteristics include inherent mass, electric charge, electron spin, mass tag, radioactive isotope, dye, bioluminescence, chemiluminescence, nucleic acid characteristics, haptens, proteins, light scattering/phase shifting characteristics, or fluorescent characteristics. As used herein, the phrase “same detectable characteristic” includes nucleotides that are detectable because they have the same signal. The same detectable characteristic includes embodiments where nucleotides are labeled with the same type of labels, for example, A and C nucleotide may be labeled with the same type of dye, where they emit the same type of signal.

Nucleotides and primers may be labeled according to any technique known in the art. Preferred labels include radiolabels, fluorescent labels, enzymatic labels, proteins, haptens, antibodies, sequence tags, mass tags, fluorescent tags and the like. Preferred dye type labels include, but are not limited to, TAMRA (carboxy-tetramethylrhodamine), ROX (carboxy-X-rhodamine), FAM (5-carboxyfluorescein), and the like.

The primer extension reaction of the present invention can employ one or more labeled nucleotide bases. Preferably, two or more nucleotides of different bases are employed. Most preferably, the primer extension reaction of the present invention employs four nucleotides of different bases. In the most preferred embodiment all four different types of nucleotide are labeled with distinguishable labels. For example, A labeled with dR6G, C labeled with dTAMRA , G labeled with dR110 and T labeled with dROX.

Once the primer extension reaction is employed, extended and unextended primers (if any) can be separated from each other so as to identify the polymorphic site on the one or more alleles that are interrogated. Separation of nucleic acids can be performed by any methods known in the art. Some separation methods include the detection of DNA duplexes with intercalating dyes such as, for example, ethidium bromide, hybridization methods to detect specific sequences and/or separate or capture oligonucleotide molecules whose structures are known or unknown and hybridization methods in connection with blotting methods well known in the art. Hybridization methods may be combined with other separation technologies well known in the art, such as separation of tagged oligonucleotides through solid phase capture, such as, for example, capture of hapten-linked oligonucleotides to immunoaffinity beads, which in turn may bear magnetic properties. Solid phase capture technologies also includes DNA affinity chromatography, wherein an oligonucleotide is captured by an immobilized oligonucleotide bearing a complementary sequence. Specific polynucleotide tags may be engineered into oligonucleotide primers, and separated by hybridization with immobilized complementary sequences. Such solid phase capture technologies also includes capture onto streptavidin-coated beads (magnetic or nonmagnetic) of biotinylated oligonucleotides. DNA may also be separated and with more traditional methods such as centrifugation, electrophoretic methods or precipitation or surface deposition methods. This is particularly so when the extended or unextended primers are in solution phase. The term “solution phase” is used herein to refer to a homogenous or heterogenous mixture. Such a mixture may be aqueous, organic, or contain both aqueous and organic components. As used herein, the term “solution” should be construed to be synonymous with suspension in that it should be construed to include particles suspended in a liquid medium.

The polymorphic sites can be detected by any means known in the art. One method of detection of nucleotides is by fluorescent techniques. Fluorescent hybridization probes may, for example, be constructed that are quenched in the absence of hybridization to target nucleic acid sequences. Other methods capitalize on energy transfer effects between fluorophores with overlapping absorption and emission spectra, such that signals are detected when two fluorophores are in close proximity to one another, as when captured or hybridized.

Nucleotides may also be detected by, or labeled with moieties that can be detected by, a variety of spectroscopic methods relating to the behavior of electromagnetic radiation. These spectroscopic methods include, for example, electron spin resonance, optical activity or rotation spectroscopy such as circular dichroism spectroscopy, fluorescence, fluorescence polarization, absorption/emission spectroscopy, ultraviolet, infrared, visible or mass spectroscopy, Raman spectroscopy and nuclear magnetic resonance spectroscopy.

Nucleotides and analogs thereof, terminators and/or primers may be labeled according to any technique known in the art. Preferred labels include radiolabels, fluorescent labels, enzymatic labels, proteins, haptens, antibodies, sequence tags, mass tags, fluorescent tags and the like. Preferred dye type labels include, but are not limited to, TAMRA (carboxy-tetramethylrhodamine), ROX (carboxy-X-rhodamine), FAM (5-carboxyfluorescein), and the like.

The term “detection” refers to identification of a detectable moiety or moieties. The term is intended to include the ability to identify a moiety by electromagnetic characteristics, such as, for example, charge, light, fluorescence, chemiluminescence, changes in electromagnetic characteristics such as, for example, fluorescence polarization, light polarization, dichroism, light scattering, changes in refractive index, reflection, infrared, ultraviolet, and visible spectra, mass, mass:charge ratio and all manner of detection technologies dependent upon electromagnetic radiation or changes in electromagnetic radiation. The term is also intended to include identification of a moiety based on binding affinity, intrinsic mass, mass deposition, and electrostatic properties, size and sequence length. It should be noted that characteristics such as mass and molecular weight may be estimated by apparent mass or apparent molecular weight, so the terms “mass” or “molecular weight” as used herein do not exclude estimations as determined by a variety of instrumentation and methods, and thus do not restrict these terms to any single absolute value without reference to the method or instrumentation used to arrive at the mass or molecular weight.

Another method of detecting the nucleotide present at the polymorphic site is by comparison of the concentrations of free, unincorporated nucleotides remaining in the reaction mixture at any point after the primer extension reaction. Mass spectroscopy in general and, for example, electrospray mass spectroscopy, may be employed for the detection of unincorporated nucleotides in this embodiment. This detection method is possible because only the nucleotide(s) complementary to the polymorphic base is (are) depleted in the reaction mixture during the primer extension reaction. Thus, mass spectrometry may be employed to compare the relative intensities of the mass peaks for the nucleotides, Likewise, the concentrations of unlabeled primers may be determined and the information employed to arrive at the identity of the nucleotide present at the polymorphic site.

In a preferred embodiment of the invention, the invention comprises a system of generating fluorescently labeled primer extension products as part of the detection assay, employing less than 5 spectrally distinct dyes. In one embodiment, four dyes are employed, wherein one or more of the dyes can also be used to label the extension products of the control reactions and, if employed, the extension product(s) of one or more flip-back primers. In one embodiment, it is possible to also monitor the success of the PCR reaction giving rise to an amplicon target nucleic acid comprising the one or more polymorphic sites to be identified; if the PCR reaction has failed, the one or more control primers will not be extended in accordance with target nucleic acid sequences, because the target nucleic acid sequences are absent. If the PCR reaction successfully generated amplicon target nucleic acid, the one or more control primers may serve at least a dual purpose: they may be employed to identify the detection primer that may be present, and to afford a level of certainty that a signal thought to be that of an extended detection primer is, in fact, the signal of an extended detection primer as opposed to background noise. Further, in a preferred embodiment of the invention, due to the judicious selection or design of control primer sequences and/or assay conditions, the apparent abundance of signal generated by the one or more control primers and the one or more detection primers can be determined as described herein. A flip-back primer may also be employed, either as a separate primer or as a feature of a control primer.

Most preferably, primer extension products are separated and identified by capillary gel electrophoreses wherein a fluorescence detector is employed to identify primer extension products labeled with fluorescent terminating nucleotides. In this most preferred embodiment, extended primers bearing fluorescent labels are separated by their mass:charge ratio. However, many separation and detection methods are known to those skilled in the art, and the invention herein is amenable to a wide variety of detection and separation protocols once this disclosure is in the hands of one skilled in the art. A primary advantage of the invention is the variety of detectable characteristics and tags that may be placed on the detection and/or control and/or flip-back primers to aid in their separation and/or detection. Indeed, in the absence of tags, the primers of the invention may be separated, detected, and/or identified by their inherent physical characteristics or behavior, as is known to those skilled in the art.

Preferred separation methods employ exposing any extended and unextended primers to a solid support. Solid supports include arrays. The term “array” is used herein to refer to an ordered arrangement of immobilized biological molecules at a plurality of positions on a solid, semi-solid, gel or polymer phase. This definition includes phases treated or coated with silica, silane, silicon, silicates and derivatives thereof, plastics and derivatives thereof such as, for example, polystyrene, nylon and, in particular, polystyrene plates, glasses and derivatives thereof, including derivatized glass, glass beads, controlled pore glass (CPG). Immobilized biological molecules includes oligonucleotides that may include other moieties, such as tags and/or affinity moieties. The term “array” is intended to include and be synonymous with the terms “chip,” “biochip,” “biochip array,” “DNA chip,” “RNA chip,” “nucleotide chip,” and “oligonucleotide chip.” All these terms are intended to include arrays of arrays, and are intended to include arrays of biological polymers such as, for example, oligonucleotides and DNA molecules whose sequences are known or whose sequences are not known.

Preferred arrays for the present invention include, but are not limited to, addressable arrays including an array as defined above wherein individual positions have known coordinates such that a signal at a given position on an array may be identified as having a particular identifiable characteristic. The terms “chip,” “biochip,” “biochip array,” “DNA chip,” “RNA chip,” “nucleotide chip,” and “oligonucleotide chip,” are intended to include combinations of arrays and microarrays. These terms are also intended to include arrays in any shape or configuration, 2-dimensional arrays, and 3-dimensional arrays.

One particularly preferred array is the GenFlex™ Tag Array, from Affymetrix, Inc., that is comprised of capture probes for 2000 tag sequences. These are 20 mers selected from all possible 20 mers to have similar hybridization characteristics and at least minimal homology to sequences in the public databases.

Another preferred array is the addressable array that has sequence tags that complement the 5′ tags of detection, control, and flip-back primers. These complementary tags are bound to the array at known positions. This type of tag hybridizes with the array under suitable hybridization conditions. By locating the bound primer in conjunction with detecting one or more extended primers, the nucleotide identity at the polymorphic site can be determined.

In one preferred embodiment of the present invention, the target nucleic acid sequences are arranged in a format that allows multiple simultaneous detections (multiplexing), as well as parallel processing using oligonucleotide arrays.

In another embodiment, the present invention includes virtual arrays where extended and unextended primers are separated on an array where the array comprises a suspension of microspheres, where the microspheres bear one or more capture moieties to separate the uniquely tagged primers. The microspheres, in turn, bear unique identifying characteristics such that they are capable of being separated on the basis of that characteristic, such as for example, diameter, density, size, color, and the like.

Having now generally described the invention, the same may be more readily understood through the following reference to the following examples, which are provided by way of illustration and are not intended to limit the present invention unless specified.

### EXAMPLES

### Example 1

Four SNPs of commercial interest lie within the coding region of the ovine PrP gene (the sequence of which is available at GENBANK accession number M31313, and is hereby incorporated by reference) and these may be assayed by multiplexed chain-terminating primer extension. As these SNPs lie in close proximity to one another, they can be assayed from a single PCR amplicon of 310 bp. This amplicon provides the target for four detection primers, each of which abut at the 3′ end one of the four SNPs of interest. There is however a significant amount of invariant DNA also represented on the 310 bp amplicon, and this invariant DNA can be used as the target for control primers which extend against invariant bases, and so generate predictable products, irrespective of the bases present at the SNP sites. Through judicious choice of the control and detection primer sequences, it has been possible to develop a single tube assay which interrogates the SNPs, and generates four labeled controls which flank the labeled detection primers. Two of the controls migrate under electrophoresis with an apparent mass smaller than all of the possible labeled detection primers. These controls both target the same core DNA sequence within the 310 bp amplicon, and interrogate the same invariant base. They differ only in the 5′ terminus, which is extended by two T bases in 50% of the primers which anneal to the target sequence. Two further controls migrate with a larger apparent mass than the detection primers. These are generated by two control primers that target another section of invariant sequence within the 310 bp sequence, and differ only in that one is two T bases longer than the other, this extension again being an addition to the 5′ terminus. Extension of any control primer results in the incorporation of a G, which carries a fluorescent dye that returns a blue signal under laser illumination. Flanking the labeled detection primer products in this way allows a Local Southern sizing algorithm to be applied to precisely size the labeled detection primer products.

The generation of labeled control extension products has enabled us to develop an automated calling software which assesses the quality of the signal generated from the controls before attempting to assess the labeled detection primer products.

Due to partial self complementarity of the control primers which generate the larger of the control products, these primers will self extend in the absence of PCR amplicon, providing a method of assessing a failure to generate a scorable profile as being due to PCR failure, or primer extension failure.

### Example 2

### Template Preparation

Template is prepared from ovine blood by alkali lysis treatment of a white blood cell pellet, followed by neutralization and dilution of the extract. A 6 microliter PCR reaction is constructed of 3 microliters extracted template (˜5 ng template) +3 microliters Mastermix [2×Gold Buffer, (ABI, Foster City, Calif.), 4 MM MgCl₂, 400 micromolar dNTP, 200 micrograms/ml heat inactivated BSA, 400 nM initial amplification primer I (CAAGGTGGTAGCCACAGTCAGTGGAACAAG) (SEQ. ID No. 1), 400 nM initial amplification primer II (CCTTGGTGGTGGTGGTGACTGTGTGTTG) (SEQ. ID NO. 2) and 0.025 units Taq Gold DNA polymerase (ABI, Foster City, Calif.). 32 cycles of PCR are performed, following the program: [(94.0° C., 11 minutes)×1, (94.0° C., 30 seconds; 64° C., 1 min; 72° C., 30 seconds)×32 cycles, (25° C. soak)].

### Example 3

### EXO/SAP Digestion

In order to remove the unincorporated nucleotides and primers, the 6 microliters of PCR product is treated with 5 units of SAP (USB) and 2 units of EXO I (NEB), and incubated at 37° C. for 1 hour before neutralizing the enzymes by raising the temperature to 72° C. for 15 minutes.

### Example 4

### Primer Extension

Two and a half microliters of the EXO/SAP digested amplification product is combined with 2.5 microliters of SNaPshot™ (ABI, Foster City, Calif.) reaction mix, which contains TaqFS DNA polymerase and fluorescently labeled ddNTPs, in addition to eight extension primers specifically designed for this assay: four controls (targeted against two invariant bases, both G incorporations) and four extension primers (targeted against the four variable SNP positions). The sequences of the extension primers are as follows:

Control primers:

| | |
|---|---|
| TCATGTGGCAGGAGCTGCTGCA [23 bp (+G) control] | (SEQ. ID NO. 3) |
| TTTCATGTGGCAGGAGCTGCTGCA [25 bp (+G) control] | (SEQ. ID NO. 4) |
| TTTTTTCCTCATAGTCATTGCCAAAATGTATAAGA [36 bp (+G) control] | (SEQ. ID NO. 5) |
| TTTTTTTTCCTCATAGTCATTGCCAAAATGTATAAGA [38 bp (+G) control] | (SEQ. ID NO. 6) |

Underscored bases indicate differences between the paired primers that target the same core sequence. The size indicated is that after the incorporation of an invariant G base.

Detection Primers:

(SEQ. ID NO. 7)

TGGTGGCTACATGCTGGGAAGTG [136F, C/T]

(SEQ. ID NO. 8)

TGGTTGGGGTAACGGTACATGTTTTCA [154R, C/T]

(SEQ. ID NO. 9)

CAACCAAGTGTACTACAGACCAGTGGATC [171-1F, G/A]

(SEQ. ID NO. 10)

CAGTCATGCACAAAGTTGTTCTGGTTACTATA [171-2R, C/A]

Each primer targets a different SNP, named in parenthesis after the sequence, together with the SNP type. These primers are present at varying concentration in the final 5 microliter extension reaction, ranging from 4 fmol/microliter to 16 fmol/microliter. These low levels of the various extension primers promote even signal intensity where the degree of target amplicon generated by the initial PCR may vary. Primer extension is performed over 25 cycles of: [(94° C., 10 sec), (54° C., 40 sec), (60° C., 20 sec)].

### Example 5

### CIP Digestion

After the primer extension reaction has been completed, the product is treated with 1 unit CIP (NEB) to neutralize the unincorporated fluorescently labeled ddNTPs prior to electroinjection on a capillary electrophoresis instrument.

### Example 6

The assay described returns very clean electropherograms (see for example FIG. 7) which have the following characteristics: The control primers extend against their targets to incorporate a G base, which carries a blue fluorescent dye. These controls are typically well balanced, and act as a reference point for the interpretation of the detection primer extension products. In the absence of target amplicon, the control primers which generate the 36 bp and 38 bp products have partial self complementarity (see FIG. 6) and act as flip-back primers, extending against themselves to incorporate a G base. This results in two blue peaks upon electrophoresis where there has been PCR failure. This proves to be a useful feature, as it indicates where during the assay the failure has occurred. Had the failure been at the primer extension stage, there would have been no detectable signal at all.

A method is described which enables the interrogation of polymorphic bases in a multiplex primer extension reaction. As an integral part of the primer extension assay, control primers are added which will extend to incorporate an invariant base, generating a predictable product. These control extension products enable the precise sizing of the extended detection primers, and permit assessment of the level of success of the assay in terms of amount of signal generated. This can be related to the success of the assay at both the PCR amplicon generation stage, and at the primer extension stage. Permitting the control primers to have partial self complementarity at the 3′ end of the primers results in a low level of self extension, and this is of utility in circumstances where the PCR reaction has failed to generate adequate amplicon for the assay to proceed optimally.

Extension of the existing assay to other assays must account for circumstances where no suitable target for the control primers exists within the amplicon generated to amplify the polymorphic region. In situations such as this the utility of generating an artificial target for the control primers becomes apparent. These controls could have very well characterized physical properties, and could be generic, with the same control sequences being used in different assays to the same effect.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of identifying one or more nucleotide bases of a target nucleic acid sequence, comprising:
providing the target nucleic acid sequence having a variant nucleotide base and an invariant nucleotide base, providing a control primer capable of hybridizing immediately adjacent to the invariant nucleotide base of the target nucleic acid sequence, providing a detection primer capable of hybridizing immediately adjacent to a variant nucleotide base of the target nucleic acid sequence;
allowing the control primer and the detection primer to hybridize to the target nucleic acid sequence;
extending the control primer and the detection primer by one or more nucleotide bases in the presence of a polymerizing agent under suitable conditions to allow primer extension to occur;
separating the control primer from the detection primer; and
identifying one or more nucleotide bases of the target nucleic acid sequence by detecting any extended control and detection primers and separating the extended detection primer from the extended control primer to ensure primer extension has occurred, thereby identifying one or more nucleotide bases of the target nucleic acid sequence.

2. A method according to claim 1, wherein the target nucleic acid sequence capable of hybridizing with the control primer is on a separate nucleic acid molecule than the target nucleic acid sequence capable of hybridizing with the detection primer.

3. A method according to claim 1, wherein the control primer and the detection primer are extended by one or more labeled nucleotide bases, and are capable of being detected by a characteristic selected from the group consisting of mass, apparent mass, molecular weight, apparent molecular weight, a combination or ratio of mass and charge, number of bases, magnetic resonance, spectrophotometry, fluorometry, electric charge, polarimetry, light scattering, luminescence, and antigen-antibody interaction.

4. A method according to claim 1, wherein the control primer bears a characteristic distinguishing it from the detection primer.

5. A method according to claim 1, wherein the control primer is a flip-back primer.

6. A method according to claim 1, further comprising a flip-back primer capable of hybridizing immediately adjacent to an invariant nucleotide base of the target nucleic acid sequence.

7. A method according to claim 1, wherein the control primer and the detection primer are extended by a chain terminator.

8. A method according to claim 7, wherein the chain-terminator comprises a dideoxynucleotide or an acyclo terminator.

9. A method according to claim 1, wherein two or more of the control primers are extended.

10. A method according to claim 7, wherein the chain terminator bears a detectable moiety.

11. A method according to claim 3, wherein each of the one or more labeled nucleotides bear a different label.

12. A method of monitoring a primer extension reaction or a reaction that generates a target nucleic acid, comprising:
providing the target nucleic acid sequence having a variant nucleotide base and an invariant nucleotide base, providing a control primer capable of hybridizing immediately adjacent to the invariant nucleotide base of the target nucleic acid sequence, providing a detection primer capable of hybridizing immediately adjacent to the variant nucleotide base of the target nucleic acid sequence;
allowing the control primer and the detection primer to hybridize to the target nucleic acid sequence;
extending the control primer and the detection primer by one or more nucleotide bases in the presence of a polymerizing agent under suitable conditions to allow primer extension to occur;
separating the control primer and the detection primer from one another; and
identifying one or more nucleotide bases of the target nucleic acid sequence by detecting any extended control primer and detection primer and separating the extended detection primer from the extended control primer to ensure primer extension has occurred, and determining the identity of the nucleotide added to the detection primer and the control primer, thereby identifying monitoring the primer extension reaction.

13. A method according to claim 12, wherein the target nucleic acid sequence capable of hybridizing with the control primer is on a separate nucleic acid molecule than the target nucleic acid sequence capable of hybridizing with the detection primer.

14. A method according to claim 12, wherein the control primer and the detection primer are extended by one or more labeled nucleotide bases, and are capable of being detected by a characteristic selected from the group consisting of mass, apparent mass, molecular weight, apparent molecular weight, a combination or ratio of mass and charge, number of bases, magnetic resonance, spectrophotometry, fluorometry, electric charge, polarimetry, light scattering, luminescence, and antigen-antibody interaction.

15. A method according to claim 12, wherein the control primer bears a characteristic distinguishing it from the detection primer.

16. A method according to claim 12, wherein the control primer is a flip-back primer.

17. A method according to claim 12, wherein the control primer and the detection primer are extended by a chain terminator.

18. A method according to claim 17, wherein the chain terminator comprises a dideoxynucleotide or an acyclo terminator.

19. A method according to claim 12, wherein two or more of the control primers are extended.

20. A method according to claim 17, wherein the terminator bears a detectable moiety.

21. A method according to claim 14, wherein each of the one or more labeled nucleotides bear a different label.

22. A method of identifying a product of a primer extension reaction, comprising:
providing two or more control primers, one or more target nucleic acid sequences and one or more detection primers, wherein the detection primer is capable of hybridizing to an invariant nucleotide sequence immediately adjacent to a polymorphic site on a target nucleic acid sequence or its complement, and wherein both of the one or more control primers hybridize to an invariant sequence on the one or more target nucleic acid sequences that differs from the invariant sequence to which the one or more detection primers hybridize;
allowing the one or more control primers and the one or more detection primers to hybridize to one or more target nucleic acid sequences;
extending the one or more control primers and the one or more detection primers in the presence of one or more labeled nucleotide bases, in the presence of a polymerizing agent, under conditions sufficient to allow primer extension to occur;
separating the control primers from the one or more detection primers; and
detecting the one or more detection primers by separating the one or more detection primers from the one or more control primers, thereby identifying the product of the primer extension reaction.

23. A method according to claim 22, wherein the target nucleic acid sequence capable of hybridizing with the control primer is on a separate nucleic acid molecule than the target nucleic acid sequence capable of hybridizing with the detection primer.

24. A method according to claim 22, wherein the control primer and the detection primer are extended by one or more labeled nucleotide bases, and are capable of being detected by a characteristic selected from the group consisting of mass, apparent mass, molecular weight, apparent molecular weight, a combination or ratio of mass and charge, number of bases, magnetic resonance, spectrophotometry, fluorometry, electric charge, polarimetry, light scattering, luminescence, and antigen-antibody interaction.

25. A method according to claim 22, wherein the control primer bears a characteristic distinguishing it from the detection primer.

26. A method according to claim 22, wherein the control primer is a flip-back primer.

27. A method according to claim 22, wherein the control primer and the detection primer are extended by a chain terminator.

28. A method according to claim 27, wherein the chain terminator comprises a dideoxynucleotide or an acyclo terminator.

29. A method according to claim 22, wherein two or more of the control primers are extended.

30. A method according to claim 27, wherein the terminator bears a detectable moiety.

31. A method according to claim 24, wherein each of the one or more labeled nucleotides bear a different label.

32. A method of monitoring a primer extension reaction, comprising:
amplifying a target nucleic acid sequence from a nucleic acid molecule of interest, in the presence of a polymerizing agent under suitable conditions for amplification to occur, wherein a pair of amplification primers capable of hybridizing to invariant regions of the nucleic acid molecule of interest are employed, and wherein the pair of amplification primers bear at their 5′ ends an invariant tag sequence comprising an invariant base wherein the invariant tag sequence is incapable of hybridizing to the nucleic acid molecule of interest, such that the invariant tag sequence comprising an invariant base is incorporated into a an amplified nucleic acid molecule comprising the target nucleic acid;
providing a control primer capable of hybridizing immediately adjacent to the invariant base of the invariant tag sequence in the amplified target nucleic acid, and providing a detection primer capable of hybridizing immediately adjacent to a variant nucleotide base of the amplified target nucleic acid;
allowing the control primer and the detection primer to hybridize to the amplified target nucleic acid sequence;
extending the control primer and the detection primer by one or more nucleotide bases in the presence of a polymerizing agent under suitable conditions to allow primer extension to occur;
separating the control primer from the detection primer; and
identifying one or more nucleotide bases of the target nucleic acid sequence by detecting any extended control and detection primers and separating the extended detection primer from the extended control primer to ensure primer extension has occurred, thereby identifying one or more nucleotide bases of the target nucleic acid sequence.

33. A method according to claim 32, wherein the target nucleic acid sequence capable of hybridizing with the control primer is on a separate nucleic acid molecule than the target nucleic acid sequence capable of hybridizing with the detection primer.

34. A method according to claim 32, wherein the control primer and the detection primer are extended by one or more labeled nucleotide bases, and are capable of being detected by a characteristic selected from the group consisting of mass, apparent mass, molecular weight, apparent molecular weight, a combination or ratio of mass and charge, number of bases, magnetic resonance, spectrophotometry, fluorometry, electric charge, polarimetry, light scattering, luminescence, and antigen-antibody interaction.

35. A method according to claim 32, wherein the control primer bears a characteristic distinguishing it from the detection primer.

36. A method according to claim 32, wherein the control primer is a flip-back primer.

37. A method according to claim 32, further comprising a flip-back primer capable of hybridizing immediately adjacent to an invariant nucleotide base of the target nucleic acid sequence.

38. A method according to claim 32, wherein the control primer and the detection primer are extended by a chain terminator.

39. A method according to claim 38, wherein the chain-terminator comprises a dideoxynucleotide or an acyclo terminator.

40. A method according to claim 32, wherein two or more of the control primers are extended.

41. A method according to claim 38, wherein the chain-terminator bears a detectable moiety.

42. A method according to claim 33, wherein each of the one or more labeled nucleotides bear a different label.


SEQUENCE LISTING (10 sequences)

SEQ ID NO: 1 - DNA, 30 bp, Artificial Sequence
  Primer - organism matches to Ovis aries
caaggtggtagccacagtcagtggaacaag

SEQ ID NO: 2 - DNA, 28 bp, Artificial Sequence
  Primer - organism matches to Ovis aries
ccttggtggtggtggtgactgtgtgttg

SEQ ID NO: 3 - DNA, 22 bp, Artificial Sequence
  Primer - organism matches to Ovis aries
tcatgtggcaggagctgctgca

SEQ ID NO: 4 - DNA, 24 bp, Artificial Sequence
  Primer - organism matches to Ovis aries
tttcatgtggcaggagctgctgca

SEQ ID NO: 5 - DNA, 35 bp, Artificial Sequence
  Primer - organism matches to Ovis aries
ttttttcctcatagtcattgccaaaatgtataaga

SEQ ID NO: 6 - DNA, 37 bp, Artificial Sequence
  Primer - organism matches to Ovis aries
ttttttttcctcatagtcattgccaaaatgtataaga

SEQ ID NO: 7 - DNA, 23 bp, Artificial Sequence
  Primer - organism matches to Ovis aries
tggtggctacatgctgggaagtg

SEQ ID NO: 8 - DNA, 27 bp, Artificial Sequence
  Primer - organism matches to Ovis aries
tggttggggtaacggtacatgttttca

SEQ ID NO: 9 - DNA, 29 bp, Artificial Sequence
  Primer - organism matches to Ovis aries
caaccaagtgtactacagaccagtggatc

SEQ ID NO: 10 - DNA, 32 bp, Artificial Sequence
  Primer - organism matches to Ovis aires
cagtcatgcacaaagttgttctggttactata